(19) United States
(12) Patent Application Publication
(10) Pub. No.: US 2010/0151466 A1
(43) Pub. Date: Jun. 17, 2010

(54) DIAGNOSIS AND TREATMENT OF CERVICAL CANCER

(75) Inventor: Richard Schlegel, Rockville, MD (US)

Correspondence Address: WOLF GREENFIELD & SACKS, P.C., 600 ATLANTIC AVENUE, BOSTON, MA, 02210-2206, US

(73) Assignee: Georgetown University, Washington, DC (US)

(21) Appl. No.: 12/575,431

(22) Filed: Oct. 7, 2009

Related U.S. Application Data
(63) Continuation of application No. 12/008,532, filed on Jan. 11, 2008, now abandoned.
Application No. 12/008,532 is a continuation of application No. 10/565,021, filed on Aug. 29, 2006, now abandoned.
(60) Provisional application No. 60/488,344, filed on Jul. 18, 2003.

(51) Int. Cl. C12Q 1/68; C12Q 1/02; C12Q 1/48

(52) U.S. Cl. 435/6; 435/29; 435/15

(57) ABSTRACT

In certain aspects, the invention relates to methods of diagnosing cervical cancer by using a combination of certain biomarkers such as hTERT, IGFBP-3, transferrin receptor, beta-catenin, Myc-HPV E6 interaction, HPV E7, and telomere length. In other aspects, the invention relates to methods of detecting immortalization of cervical cells by using a combination of certain biomarkers. In yet other aspects, the invention relates to methods of classifying the grade of a cervical lesion for diagnostic and prognostic purposes in a female. In further aspects, the invention relates to methods of treating cervical cancer by administering a therapeutic agent that targets one or more of these biomarkers.

### RELATED APPLICATIONS

This application claims the benefit of the filing date of U.S. Provisional Application No. 60/488,344, filed Jul. 18, 2003. The entire teachings of the referenced Provisional Application are incorporated herein by reference in their entirety.

### FUNDING

Work described herein was funded, in part, by National Cancer Institute Grant RO1CA53371. The United States government has certain rights in the invention.

### BACKGROUND

Cervical cancer is the second most common cancer in women worldwide with approximately 400,000 new cases being diagnosed each year despite the existence of screening methods. Infection with human papilloma virus (HPV) is the cause of almost every case of cervical cancer. Infection with human papilloma viruses is a common sexually transmitted infection; more than 50 different viral types are found as human genital infections. However, only 10-15 types are able to cause cervical cancer and by far the most common of these are HPV-16 and HPV-18. These viruses encode transforming oncoproteins E6 and E7 and play a key role in human cervical cancer.

There are a number of known methods for diagnosing cervical cancer. Initial large scale screening relies mainly on cytological screening of cervical smear samples. Smear samples are taken using routine procedures, and analyzed for abnormal cell morphology. Samples are then classified in a number of categories. However, cytological screening is not reliable and often gives inaccurate results. In individual cases, more invasive procedures are often necessary to establish a firm diagnosis. Colposcopy review may be carried out, and in cases where lesions are detected or suspected, a biopsy may be taken for further more accurate analysis.

HPV-16 and HPV-18 can be detected in women with undetectable or minimal cervical abnormality. Thus, cellular factors may therefore regulate the progression of HPV induced cervical transformation. For example, it has been suggested that women with a p53 tumor suppressor protein having an arginine rather than a proline residue at position 72 show an enhanced risk of cervical cancer because the HPV E-6 protein can cause more efficient degradation of the arginine-containing form of p53, thereby neutralizing its tumor suppressor function more effectively.

Clearly, there is a need for additional approaches to diagnosing and treating cervical cancer which is a significant public health problem.

### SUMMARY OF THE INVENTION

The present invention relates to methods of diagnosing or aiding in the diagnosis of cervical diseases or conditions, including cervical cancer, cervical precancer, or immortalization of cervical cells, by using a panel of biomarkers. The present invention also relates to methods of treating cervical diseases (e.g., cervical cancer) by targeting one or more of these biomarkers.

In one embodiment, the invention provides a method of diagnosing or aiding in the diagnosis of cervical cancer in a female, who may be of any age (e.g., child or adult). For example, the female (e.g., girl or woman) is suspected of having or is known to have cervical cancer (e.g., associated with HPV infection). Alternatively, the diagnostic method can be carried out in any woman, such as during or in conjunction with routine (regular) healthcare screenings (e.g., periodic physical examinations). Such method comprises analyzing the status of at least two of the following biomarkers: human telomerase reverse transcriptase (hTERT), insulin-like growth factor binding protein 3 (IGFBP-3), transferrin receptor, beta-catenin, Myc-HPV E6 interaction, HPV E7, and telomere length, in cervical cells of the female. As used herein, the status of each biomarker is referred to as follows.

If the biomarker is hTERT, IGFBP-3, transferrin receptor or HPV E7, the status to be assessed is the expression level of the biomarker. Thus, in this method, the expression level of the biomarker is analyzed. Preferably, the expression level of HPV E7 is analyzed by flow cytometry. Increased expression level of the biomarker relative to an appropriate control level (e.g., obtained from a healthy female) indicates that the female has cervical cancer or is at increased risk of developing cervical cancer.

If the biomarker is beta-catenin, the status to be assessed is the level and localization of beta-catenin in the cytoplasm and/or nucleus. Thus, in this method, the level and localization of beta-catenin are analyzed. Increased level of beta-catenin in the cytoplasm and/or nucleus relative to an appropriate control level (e.g., obtained from a healthy female) indicates that the female has cervical cancer or is at increased risk of developing cervical cancer.

If the biomarker is Myc-HPV E6 interaction, the status to be assessed is the association between Myc and HPV E6. Thus, in this method, the association between Myc and HPV E6 is analyzed. Association between Myc and HPV E6 indicates that the female has cervical cancer or is at increased risk of developing cervical cancer. Certain aspects of the invention relate to use of Myc modifications (e.g., phosphorylation) or mutations in Myc as biomarkers in the methods of the present invention.

If the biomarker is telomere length, the status to be assessed in this method is the telomere length. Increased telomere length relative to an appropriate control length (e.g., obtained from a healthy female) indicates that the female has cervical cancer or is at increased risk of developing cervical cancer.

In another embodiment, the invention provides a method of diagnosing or aiding in the diagnosis of cervical cancer in a female. Such method comprises analyzing the status of at least two biomarkers in cervical cells of the female. One biomarker is Myc-HPV E6 interaction, while a second biomarker is selected from the group consisting of: hTERT, IGFBP-3, transferrin receptor, beta-catenin, HPV E7, and telomere length. The status of each biomarker is described above.

In still another embodiment, the invention provides a method of detecting immortalization of cervical cells in a female, who may be of any age (e.g., child or adult). For example, the female is suspected of having or is known to have cervical cancer (e.g., associated with HPV infection). Alternatively, the diagnostic method can be carried out in any woman, such as during or in conjunction with routine (regular) healthcare screenings (e.g., periodic physical examinations). Such method comprises analyzing the status of at least two of the following biomarkers: hTERT, IGFBP-3, transferrin receptor, beta-catenin, Myc-HPV E6 interaction, HPV E7, and telomere length, in cervical cells of the female.

In a further embodiment, the invention provides a method of classifying the grade of a cervical lesion for diagnostic and/or prognostic purposes in a female. Such method comprises: (a) determining the status of one (or more) biomarker in a cervical cell of a female to provide an individual biomarker diagnostic for cervical lesions, wherein the biomarker is selected from the group consisting of: hTERT, IGFBP-3, transferrin receptor, beta-catenin, Myc-HPV E6 interaction, HPV E7, and telomere length, and combinations thereof; (b) comparing the status of the individual biomarker with a biomarker reference panel (e.g., a reference panel including mean values of the status for the biomarker constituents of the panel); and (c) classifying a cervical lesion for the female by said comparison. Preferably, the biomarker reference panel of the method comprises a constituent panel developed using cervical cancer, high grade cervical lesion, low grade cervical lesion, and control group populations.

In yet another embodiment, the invention provides a method of treating a female suffering from cervical cancer (e.g., associated with HPV infection). Such method comprises administering to the female a therapeutically effective amount of an agent which targets and blocks or decreases the function (e.g., expression or activity) of one or more of the biomarkers. In one case, the agent blocks interaction between Myc and HPV E6. In other cases, the agent blocks or reduces the expression level of hTERT, IGFBP-3, transferrin receptor, beta-catenin, HPV E6, or HPV E7. In a particular case, the agent blocks signaling through the beta-catenin pathway. Exemplary therapeutic agents in such methods include, but are not limited to, small molecules, polypeptides, antibodies, and nucleic acids. In specific embodiments, the present invention contemplates the use of antisense nucleic acids or RNA interference (RNAi) nucleic acids to block or reduce gene expression of one or more of the above biomarkers.

In a further embodiment, the present invention provides a method of preventing the onset of cervical cancer (e.g., associated with HPV infection) or reducing the extent to which it occurs in a female. Such method comprises administering to the female an effective amount of an agent which targets and blocks or decreases the function (e.g., expression or activity) of one or more of the biomarkers. The agent is effective to prevent the onset of cervical cancer or reduce the extent to which it occurs. In one case, the agent blocks interaction between Myc and HPV E6. In other cases, the agent blocks or reduces the expression level of hTERT, IGFBP-3, transferrin receptor, beta-catenin, HPV E6, or HPV E7. In a particular case, the agent blocks signaling through the beta-catenin pathway. Exemplary therapeutic agents in such methods include, but are not limited to, small molecules, polypeptides, antibodies, and nucleic acids. In specific embodiments, the present invention contemplates the use of antisense nucleic acids or RNA interference (RNAi) nucleic acids to block or reduce gene expression of one or more of the above biomarkers.

### DETAILED DESCRIPTION OF THE INVENTION

In one embodiment, the invention provides a method of diagnosing or aiding in the diagnosis of cervical cancer in a female, who may be of any age (e.g., child or adult). For example, the female is suspected of having or is known to have cervical cancer (e.g., associated with HPV infection). Alternatively, the diagnostic method can be carried out in any woman, such as during or in conjunction with routine (regular) healthcare screenings (e.g., periodic physical examinations). Such method comprises analyzing the status of at least two of the following biomarkers: hTERT, IGFBP-3, transferrin receptor, beta-catenin, Myc-HPV E6 interaction, HPV E7, and telomere length, in cervical cells of the female. The status of each biomarker is described above. Discoveries relating to these biomarkers are described below under the section “Exemplary Biomarkers for Cervical Cancer.”

In another embodiment, the invention provides a method of diagnosing or aiding in the diagnosis of cervical cancer in a female. Such method comprises analyzing the status of at least two biomarkers in cervical cells of the female. One biomarker is Myc-HPV E6 interaction, while a second biomarker is selected from the group consisting of hTERT, IGFBP-3, transferrin receptor, beta-catenin, HPV E7, and telomere length. Alternatively, one biomarker can be selected from Myc modifications (e.g., phosphorylation) and mutations in Myc.

In still another embodiment, the invention provides a method of detecting immortalization of cervical cells in a female. Such method comprises analyzing the status of at least two of the following biomarkers: hTERT, IGFBP-3, transferrin receptor, beta-catenin, Myc-HPV E6 interaction, HPV E7, and telomere length, in cervical cells of the female.

In yet another embodiment, the invention provides a method of treating a female suffering from cervical cancer (e.g., associated with HPV infection), and a method of preventing the onset of cervical cancer or reducing the extent to which it occurs in a female. Such methods comprise administering to the female a therapeutically effective amount of an agent which targets and blocks or decreases the function (e.g., expression or activity) of one of the biomarkers. In one case, the agent blocks interaction between Myc and HPV E6. In other cases, the agent blocks or reduces the level of expression of hTERT, IGFBP-3, transferrin receptor or beta-catenin. In a particular case, the agent blocks signaling through the beta-catenin pathway. Exemplary therapeutic agents in such methods include, but are not limited to, small molecules, polypeptides, antibodies, and nucleic acids. In specific embodiments, the present invention contemplates the use of antisense nucleic acids or RNA interference (RNAi) nucleic acids to block or reduce gene expression of one or more of the above biomarkers.

### Exemplary Biomarkers for Cervical Cancer

The present invention contemplates use of certain biomarkers in diagnosing and treating cervical cancer. In specific embodiments, these biomarkers can be used in detecting immortalization of cervical cells. Examples of biomarkers for the present invention include, but are not limited to, hTERT, IGFBP-3, transferrin receptor, beta-catenin, Myc (e.g., Myc-HPV E6 interaction, a myc modification or a mutation in Myc), HPV E7, and telomere length.

1) hTERT

Telomerase activity is detected in more than 90% of immortalized and cancer cells but absent in most normal somatic cells (Kim et al., 1994, Science, 266:2011-2015; Meyerson et al., 1997, Cell, 90:785-795), suggesting that telomerase activation is an important event during the process of immortalization and malignant transformation. Telomerase activity is closely associated with the expression of the telomerase catalytic subunit, hTERT. The expression of hTERT RNA is detected at high levels in tumor tissues and tumor-derived cell lines but not in normal adjacent tissues or primary cells (Ramakrishnan et al., 1998, Cancer Res., 58:622-625; Takakura et al., 1998, Cancer Res., 58:1558-1561). It is suggested that hTERT is the rate-limiting determinant of enzymatic activity of human telomerase and that upregulation of hTERT might be a critical event in the development of human cancers.

Over-expression of hTERT is responsible for the increase in cellular telomerase activity (see, e.g., Veldman et al., 2001, J Virol., 75:4467-72; Yuan et al., 2002, J Virol., 76:10685-91). This activity is essential for cell immortalization and for cancer progression. More importantly, hTERT expression exhibits a very large increase during immortalization, independent of HPV gene expression (Baege et at, 2002, Am J Pathol., 160:1251-7). Thus, this increased expression of hTERT can be used to confirm or detect immortalization, an important step in the progression of HPV-infected cells to the immortal state, and can be used to differentiate between cervical cells which are simply infected but not immortalized by HPV and those cells that are infected and immortalized by HPV. This marker alone, or in combination with other markers described herein, provides a diagnostic tool for detecting cervical cancer cells, such as those cells which have progressed from the HPV-infected state to the immortal state. Further, hTERT can be a therapeutic target for cervical cancer.

### 2) IGFBP-3

Insulin-like growth factor binding protein 3 (IGFBP-3), the most abundant IGFBP in human serum, is synthesized mainly by hepatic Kupffer cells and binds over 90% of circulating IGF, resulting in a prolonged half-life of IGF (Baxter et al., 1989, Prog. Growth Factor Res., 1:49-68). IGFBP-3 is also produced locally by a variety of normal and tumor cells. The biological functions of IGFBP-3, aside from being the major binding protein for IGF-1, are complex and remain poorly understood. IGFBP-3 modifies the interaction of IGF-1 with its receptor (Kelly et al., 1996, Int. J. Biochem. Cell Biol., 28:619-637), and modulates IGF-1 activity by binding to the extracellular matrix and cell surfaces, possibly to yet identified receptors (Baxter et al., 2000, Am. J. Physiol. Endocrinol. Metab., 278:967-976). Furthermore, it has been suggested that IGFBP-3 may signal independently of IGF and that it can be translocated to the nucleus where it interacts with nuclear components, which remain to be identified (Baxter et al., 2001, Mol. Pathol., 54:145-148).

IGFBP-3 is over-expressed in immortalized cervical cells. Similar to hTERT, IGFBP-3 shows a large increase (˜500 fold) in expression during the process of cell immortalization (Berger et al., 2002, Am. J. Pathol., 161:603-610). In addition,

IGFBP-3 can be a positive regulator of IGF-1 signaling and appears to have an important role in sensitizing cervical cells to IGF-1. Thus, IGFBP-3 can augment the growth of cervical cells and may have a critical role in cervical cancer. In particular, IGFBP-3 alone, or in combination with other markers described herein, can potentially be used to detect cervical cancer cells. Further, IGFBP-3 can be a therapeutic target for cervical cancer.

### 3) Transferrin Receptor

Transferrin receptor is present on almost all mammalian cells. Transferrin receptor binds the major serum iron-transport protein, transferrin, and mediates cellular iron uptake by receptor-mediated endocytosis.

Cervical cancer cells over-express transferrin receptor. Over-expression of transferrin receptor can be used, alone or in combination with other markers as described herein, as a diagnostic marker for detecting cervical cancer cells. Further, transferrin receptor can be a therapeutic target for cervical cancer.

### 4) Beta-catenin

β-catenin protein functions in two independent processes: cell-cell adhesion and signal transduction (Peifer, 1997, Science, 275:1752-3). In the adherence junctions, it binds to the cytoplasmic tail of E-cadherin and mediates the interaction between the adherence junctions and actin microfilaments with α-catenin. In cells, β-catenin is localized mostly in such adherent junctions, and the free cytoplasmic β-catenin level is very low. Elevation of the free β-catenin level in the cytoplasm can be caused by mutation of β-catenin itself (Rubinfeld et al., 1997, 275:1790-2).

The conversion of immortal genital cells to the tumorigenic phenotype is accompanied by the increased expression of β-catenin in the cytoplasm and/or nucleus. This provides an important understanding of the progression of cervical cancer. β-catenin has been observed to be expressed in cervical cancer but its gene is not mutated. In addition, Applicants' findings help understand the mechanism for the increased expression of β-catenin in the cytoplasm and/or nucleus. Applicants also identified the β-catenin pathway that contributes to the conversion to cervical cancer. Therefore, the β-catenin pathway (e.g., expression and/or localization of β-catenin in the cytoplasm and/or nucleus) can be used alone, or in combination with other markers as described herein, as diagnostic biomarkers for detecting cervical cancer cells. Further, the β-catenin pathway can potentially offer several therapeutic targets (e.g., β-catenin) for cervical cancer.

### 5) Myc

Myc protein is a critical regulator of epithelial cell growth and differentiation. c-Myc, as a transcription factor, can promote cell proliferation by regulating the expression of numerous target genes. Applicants recently discovered that Myc protein is a target of the HPV E6 protein in cervical cancer. Specifically, Myc associates with HPV E6 protein and cooperatively activates the hTERT promoter (Veldman et al., 2003, Proc Natl Acad Sci U S A., 100:8211-6). Thus, Applicants suggest that Myc/E6 interaction can be used alone, or in combination with other markers as described herein, for detecting cervical cancer cells. In addition, Applicants suggest that therapeutic approaches for cervical cancer can be designed by interfering with the Myc/E6 interaction.

Optionally, Myc modifications and/or mutations can be used as possible diagnostic markers for detecting cervical cancer. For example, activities of Myc protein may be regulated by post-translational modifications which include, but are not limited to, acetylation, carboxylation, glycosylation, phosphorylation, lipidation, and acylation. To illustrate, there are two major phosphorylation sites in the N-terminal transactivation domain of Myc (Thr-58 and Ser-62) that regulate transcriptional and transforming activities of Myc (Henriksson, et al., 1993, Oncogene 8:3199-3209; Blackwood et al., 1991, Science 251:1211-1217). Thus, certain modifications of Myc (e.g., phosphorylation) may be associated with cancers such as cervical cancer. Further, it is possible to screen for and identify mutations in Myc which alter Myc activities. Thus, certain mutations in Myc may also be associated with cancers such as cervical cancer. Applicants propose that Myc modifications and/or mutations may have an important role in tumor progression and in cancer diagnosis.

### 6) HPV E7 Expression

Two oncoproteins, E6 and E7, are encoded by the high-risk HPVs. Both HPV E6 and HPV E7 can form specific complexes with tumor suppressor gene products. The HPV E7 protein binds to the retinoblastoma tumor suppressor gene product (pRB). The HPV E6 protein can associate with the p53 tumor suppressor protein. The functional inactivation of pRB and p53 by the HPV oncoproteins E7 and E6, respectively, are likely to be important steps in cervical carcinogenesis.

Applicants have recently shown that it is possible to detect expression of HPV E7 protein in cervical cancer cells by flow cytometry. This technique could be used to rapidly identify HPV-infected cells. In combination with other markers described herein, this can be the basis for a rapid assay for cervical cancer cells. For example, HPV E7 gene expression can be detected by performing polymerase chain reactions (PCR) inside of intact cells. Measurement of genetic parameters and observation of genetic properties while maintaining the integrity of the DNA or RNA in a cell is then accomplished by passing a suspension of cells through a flow cytometer wherein the properties and parameters can be measured on a cell by cell basis. Specifically, cells are first fixed by suspension in a solution comprising ultrapure formaldehyde and then removed from the solution. A polymerase is then added into the cells to amplify specific genetic material (e.g., E7). Finally, the amplified genetic material in individual cells is rapidly detected by a flow cytometer.

A flow cytometer is an instrument that will measure fluorescence of individual cells as they pass in single file through a light source (usually a laser beam). Antibodies labeled with fluorescent dyes directed against cell antigens, fluorescent dyes that label specific substrates in the cell and fluorochromes that are sensitive to ions have all been used to label specific cell populations or molecules within cells for identification and evaluation of function. Flow cytometry can also be used to sort cells.

As cells labeled with a fluorochrome attached in some way to the desired component pass through the laser beam, the fluorochrome is excited. The emission is detected orthogonally (perpendicular) to the laser beam as the light passes through a focusing lens system and spectral filters to selectively detect the desired wavelength. The light is then detected by a photomultiplier tube that integrates all the fluorescence that passes through the color bandpass filter. Nonspecific cellular fluorescence called autofluorescence appears yellow to the eye but there is a significant green component to it and this component is passed along with the green fluorescein fluorescence from fluorescein through the bandpass filter. Thus, the flow cytometer detects both the autofluorescence and the specific fluorescence from the component that is stained with fluorescein. If the fluorescence of the stained component is too low, it will not be resolved from the autofluorescence. A method to amplify the fluorescence of the desired component above the autofluorescence and other nonspecific fluorescence has been developed.

### 7) Telomere Length

Telomeres containing noncoding DNA repeats at the end of the chromosomes are essential for chromosomal stability and are implicated in regulating the replication and senescence of cells. The gradual loss of telomere repeats in cells has been linked to aging and tumor development. As described above, Applicants have evidence that hTERT is over-expressed during immortalization. Applicants also found that increased telomere length results from this increased telomerase activity. It is therefore possible to identify immortalized cells with fluorescent probes for telomere length, and may be used alone, or in combination with other markers described herein, as a diagnostic approach for cervical cancer.

Procedures and methods for measuring telomere length are known in the art and can be used in this invention. For detection of telomeric length, one may study just a particular cell type, all cells in a tissue (where various cells may be present), or subsets of cell types, and the like. The preparation of the DNA having such telomeres may be varied, depending upon how the telomeric length is to be determined. At least three methods for measuring the length of telomere repeats have been described: Southern blot analysis and quantitative fluorescence in situ hybridization using either digital fluorescence microscopy (Q-FISH) or flow cytometry (flow-FISH). See, e.g., Allshire et al., 1988, Nature, 332:656-659; de Lange et al., 1990, Mol. Cell Biol. 10:518-527; Rufer et al., 1998, Nature Biotech, 16: 743-747; and Poon et al., 1999, Cytometry, 36:267-278. Methods for measuring telomere length are also described in, for example, U.S. Pat. Nos. 6,368,789 and 6,551,774.

For example, Southern blot analysis is a multi-step method which entails: (a) cleaving purified DNA with restriction enzymes; (b) separating the DNA fragments by size on an agarose gel; (c) denaturing and transferring the DNA fragments to a membrane; (d) hybridizing the telomere with a radioactive telomere probe; (e) removing the unhybridized probe by washing the membrane; and (f) analyzing the data by autoradiography and image analysis (see, e.g., Allshire et al., 1988, Nature, 332:656-659; de Lange et al., 1990, Mol. Cell Biol. 10:518-527). In addition, several alternative methods have been described in recent years. Some, such as pulsed-field electrophoresis, slot blots, and centromere-to-telomere ratio measurements are essentially improvements to the Southern blot technique.

However, other methods, such as fluorescent in situ hybridization on metaphase chromosome spreads and flow cytometry-based fluorescent in situ hybridization, represent a new technical approach to the problem (see, e.g., Rufer et al., 1998, Nature Biotech, 16: 743-747; Poon et al., 1999, Cytometry, 36:267-278). For example, in the flow-FISH technique, a fluorescein isothiocyanate (FITC)-labeled telomere-specific peptide nucleic acid (PNA) probe is hybridized in a quantitative way to telomere repeats, followed by telomere fluorescence measurements on individual cells by flow cytometry.

### Methods of Diagnosis

In certain embodiments, the present invention provides methods of diagnosing or aiding in the diagnosis of cervical cancer in a female. In certain embodiments, the present invention relates to methods of detecting immortalization of cervical cells in a female as it is known that immortalized cervical cells have striking parallels to high-grade cervical lesions. For example, when HPV-16- or HPV-18-immortalized cervical cells are grown in raft cultures, they form structures similar to high-grade cervical lesions, characterized by a lack of stratification and differentiation, an expansion of basal-type cells throughout the epithelium, and cellular disorganization and nuclear atypia (see, e.g., Rader et at, 1990, Oncogene, 5:571-576; Pecoraro et al., 1991, Am J Pathol, 138:1-8). These methods of diagnosis comprise detecting the status of a biomarker selected from hTERT, IGFBP-3, transferrin receptor, beta-catenin, Myc-HPV E6 interaction, HPV E7, and telomere length, or combinations thereof, in cervical cells of the female.

In certain embodiments, the present invention provides methods of classifying the grade of a cervical lesion for diagnostic and/or prognostic purposes in a female. For example, such method comprises the following steps: (a) determining the status of one or more biomarkers in a cervical cell of a female to provide individual biomarkers diagnostic for cervical lesions, wherein the biomarkers are selected from the group consisting of: hTERT, IGFBP-3, transferrin receptor, beta-catenin, Myc-HPV E6 interaction, HPV E7, and telomere length; (b) comparing the status of the biomarkers with a biomarker reference panel (e.g., a reference panel including mean values of the status for the biomarker constituents of the panel); and (c) classifying a cervical lesion for the female by said comparison. Preferably, the status of the biomarker is significantly higher for a high grade cervical lesion (e.g., cervical cancer) than for a low grade cervical lesion (e.g., cervical precancer).

For example, the method of the invention can be used for classifying (or categorizing) female subjects in one of several diagnostic groups. In increasing order of severity, these groups include “negative,” “low grade squamous intraepithelial lesions (LGSIL: HPV-CIN1),” “high grade squamous intraepithelial lesions (HGSIL: CIN2-CIN3),” and “cervical cancer.” Applicant has established that presence or absence of a biomarker gene product (e.g., mRNA or protein), level of expression of a biomarker gene product, subcellular (e.g., cytoplasm or nucleus) localization or level of a biomarker gene product, interaction of a biomarker protein with its associated protein, or telomere length in a cell sample (e.g., cervical cells), may be used to give an accurate predictor of the final diagnostic group of a female from which the sample is taken. For example, presence of HPV E7 in a sample may be indicative of increased susceptibility to cervical cancer. As another example, mean values of a biomarker mRNA or protein in a sample can show statistical differences between samples from patients in each of the final diagnostic groups. To illustrate, a sample from an HGSIL patient may have significantly higher levels of hTERT, IGFBP-3, and/or transferrin receptor than one from an LGSIL patient and one from a normal (healthy) female. The phrase “significantly higher” as described herein is well within the knowledge of a skilled artisan, and will be determined empirically with reference to the particular biomarker. For example, the phrase “significantly higher” is relative to an appropriate control level (e.g., a level determined in samples from healthy females).

Optionally, the biomarker reference panel of the method comprises a constituent panel developed using cervical cancer, high grade cervical lesion, low grade cervical lesion, and control group populations. The reference panel includes one or more biomarkers identified as having diagnostic value, such as the biomarkers described in this application as well as other biomarkers for cervical cancer (e.g., E6 or E7 proteins). Optionally, each referenced biomarker constituent of the panel can have a range of values that correspond to various diagnostic groups.

The term “cervical cells” as used herein, refers to cell samples (e.g., primarily a collection of cells) from the cervix of a patient (a subject or an individual, preferably a female). One method of obtaining cells is through non-invasive means, which is defined herein as obtained without the puncturing of a patient. Examples of non-invasive means are, for example, cell samples obtained from cervical or other cell surface scrape. Patient cells can also be obtained by other means including, for example, needle biopsy or tissue biopsy.

The cervical cells used in the present invention can be preserved in a collection medium which allows for a combination of two or more assays of different characteristics related to a cell state of interest. As used herein, the assay or assays refer to a section or measurement of specific characteristics, the results of which may be combined with other such measurements of other characteristics to provide an overall assessment of a cell suspected of being affected by one or more diseases or conditions. These assays may include, for example, a combination of morphological analysis and quantitation of a particular RNA or DNA or protein whose levels provide a specific indication of the presence or progression of a disease. Alternatively, for example, the collection medium can be used to combine an assay identifying the morphology of cells in a cell sample with one or more assays identifying the HPV type involved, and, for example, identifying whether the HPV type identified is a high risk or low risk HPV type for the development of HPV-induced cell transformation and cancer.

Cervical cell samples for use in the present invention can be collected and stored in liquid medium. Examples of useful cell collection media are STM (Digene), PreservCyt® (Cytyc), and CytoRich® (Autocyte). These media (PreservCyt® and CytoRich®) were developed for the collection of cytological samples but can be adapted, for use with molecular assays.

Cervical cell samples for use in the method of the present invention can be fixed or processed in any manner consistent with the assays to be performed. For example, both cytological and molecular assays can be performed using cells fixed on a solid substrate such as, for example, a slide. The requirements of the assays to be performed will generally identify the sample processing to be used.

In further embodiments, the present invention provides a kit suitable for use in the present diagnostic methods. For example, those methods can be conveniently performed using kits that include one or more of the materials needed for the method, such as reagents and sample collection and handling materials. For example, kits can include cell collection medium, sample preserving reagents, reagents for specific detection of DNA and/or expression products (RNA or proteins) of one or more of the biomarkers (e.g., hTERT, IGFBP-3, transferrin receptor, beta-catenin, Myc-HPV E6 interaction, HPV E7, and telomere length) and sample handling containers. Useful reagents for detection of gene expression of certain biomarkers are nucleic acid probes for those genes. A kit may also contain control samples or reagents, or reagents and materials for performing other assays to be combined with the disclosed assay. In addition, the kits can contain reagents for the separation of RNA and/or DNA from other cellular components.

The present invention can be performed using devices adapted to the method. Numerous devices for performing similar assays are known and in use and can be adapted for use with the disclosed assays and method. For example, devices are known for automating all or a part of sample assays and sample handling in assays.

In certain embodiments, diagnostic methods of the present invention can include the combination with any other assays for assessing a disease or state of cells in a cell sample. For example, the subject diagnostic methods can be combined with cytological assays, histological assays, determination of the HPV type, determination of the level of HPV, assays detecting other cellular markers such as oncoproteins or tumor suppressors, or any combinations of these assays. Such assays are known and are used for the diagnosis of HPV infection or cervical diseases (e.g., cervical cancer) and assessment of the stage of the cervical disease. Results from the subject diagnostic methods and one or more additional assays can be combined to increase the reliability of any assessment, prognosis, diagnosis, or monitoring of cervical diseases. Where multiple assays point in the same prognostic or diagnostic direction, the reliability of the assessment is increased. Combined assays can be performed in any order and in any temporal relationship. For example, various assays can be performed in parallel or simultaneously. Such assays can be performed in any manner such as on the same apparatus by the same person, with different apparatus, or in the same or different locations.

For example, cytological assays for use in assessing the stage of HPV-based diseases (e.g., cervical cancer) are known and can be combined with the disclosed method. The well established Pap smear and Hematoxylin & Eosin stains (H&E) are preferred examples. The use and analysis of Pap smears and H&E stains are well-known in the art.

Methods of the present invention involve noninvasive procedures which are suitable for large scale screening of patients and are more accurate than conventional cytological screening. Further, since the subject methods can be used for predicting the final diagnostic group of a patient by classifying the grade of a cervical lesion, it is possible to select the treatment most appropriate for that patient. For example, an LGSIL patient can simply be closely monitored rather than subjected unnecessarily to the more harsh aggressive treatment appropriate for a HGSIL patient.

### Methods of Treatment

In certain embodiments, the present invention provides methods of treating a female suffering from cervical cancer, as well as methods of preventing the onset of cervical cancer in a female. As used herein, a therapeutic (therapeutic agent or therapeutic compound) that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. Thus, prevention of cervical cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. The term “treating” as used herein includes prophylaxis of the named condition or amelioration or elimination of the condition once it has been established.

In certain embodiments, methods of the invention comprise administering to the female a therapeutically effective amount of a therapeutic agent which targets one or more of the biomarkers as described above (e.g., hTERT, IGFBP-3, transferrin receptor, beta-catenin, and Myc-HPV E6 interaction). To illustrate, a therapeutic agent of the invention can block or reduce the level of expression of hTERT, IGFBP-3, transferrin receptor or beta-catenin. Alternatively, a therapeutic agent of the invention can block interaction between Myc and HPV E6. The therapeutic agents of the present invention include, but are not limited to, a polypeptide, an antibody, a small organic molecule, a peptidomimetic, and a nucleic acid.

In certain aspects, the therapeutic agents may include a polypeptide and an antibody. Such therapeutic agents can, for example, prevent the interaction between Myc or HPV E6, or block signaling through the beta-catenin pathway. Antibodies may be polyclonal or monoclonal; intact or truncated, e.g., F(ab′)2, Fab, Fv; xenogeneic, allogeneic, syngeneic, or modified forms thereof, e.g., humanized, chimeric, etc.

In certain aspects, the therapeutic agents of the present invention include a nucleic acid. In one embodiment, the invention relates to the use of antisense nucleic acid to decrease expression of one or more of the biomarkers (e.g., hTERT, IGFBP-3, transferrin receptor or beta-catenin). Such an antisense nucleic acid can be delivered, for example, as an expression plasmid which, when transcribed in the cell, produces RNA which is complementary to at least a unique portion of the cellular mRNA which encodes a biomarker polypeptide (e.g., hTERT, IGFBP-3, transferrin receptor or beta-catenin). Alternatively, the construct is an oligonucleotide which is generated ex vivo and which, when introduced into the cell causes inhibition of expression by hybridizing with the mRNA and/or genomic sequences encoding a biomarker polypeptide. Such oligonucleotide probes are optionally modified oligonucleotide which are resistant to endogenous nucleases (e.g., exonucleases and/or endonucleases), and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

In another embodiment, the invention relates to the use of RNA interference (RNAi) to effect knockdown of one or more of the biomarker genes (e.g., hTERT, IGFBP-3, transferrin receptor or beta-catenin). RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. RNAi provides a useful method of inhibiting gene expression in vitro or in vivo. RNAi constructs can comprise either long stretches of dsRNA identical or substantially identical to the target nucleic acid sequence or short stretches of dsRNA identical to substantially identical to only a region of the target nucleic acid sequence.

Optionally, the RNAi constructs contain a nucleotide sequence that hybridizes under physiologic conditions of the cell to the nucleotide sequence of at least a portion of the mRNA transcript for the gene to be inhibited (e.g., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

The subject RNAi constructs can be “small interfering RNAs” or “siRNAs.” These nucleic acids are around 19-30 nucleotides in length, and even more preferably 21-23 nucleotides in length. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group. In certain embodiments, the siRNA constructs can be generated by processing of longer double-stranded RNAs, for example, in the presence of the enzyme dicer. The combination is maintained under conditions in which the dsRNA is processed to RNA molecules of about 21 to about 23 nucleotides. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis. Methods of chemically modifying RNA molecules can be adapted for modifying RNAi constructs (see, e.g., Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

Alternatively, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci U S A, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

PCT application WO 01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

In another embodiment, the invention relates to the use of ribozyme molecules designed to catalytically cleave an mRNA transcripts to prevent translation of mRNA (see, e.g., PCT International Publication WO90/11364, published Oct. 4, 1990; Sarver et al., 1990, Science 247:1222-1225; and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art and is described more fully in Haseloff and Gerlach, 1988, Nature, 334:585-591. The ribozymes of the present invention also include RNA endoribonucleases (hereinafter “Cech-type ribozymes”) such as the one which occurs naturally in Tetrahymena thermophila (known as the IVS or L-19 IVS RNA) and which has been extensively described (see, e.g., Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216).

In a further embodiment, the invention relates to the use of DNA enzymes to inhibit expression of one or more of the biomarker gene (e.g., hTERT, IGFBP-3, transferrin receptor or beta-catenin). DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid. Briefly, to design an ideal DNA enzyme that specifically recognizes and cleaves a target nucleic acid, one of skill in the art must first identify the unique target sequence. Preferably, the unique or substantially sequence is a G/C rich of approximately 18 to 22 nucleotides. High G/C content helps insure a stronger interaction between the DNA enzyme and the target sequence. When synthesizing the DNA enzyme, the specific antisense recognition sequence that will target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462.

In certain aspects, the therapeutic agents of the present invention include a small molecule (e.g., a peptidomimetic). Examples of small molecules include, but are not limited to, small peptides or peptide-like molecules (e.g., a peptidomimetic). As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics.

As described herein, small molecule compounds may encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulfhydryl or carboxyl group. Candidate small molecule compounds can be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant, and animal extracts are available or readily produced. Additionally, natural or synthetically produced libraries and compounds can be modified through conventional chemical, physical, and biochemical means. Known pharmacological agents may be subjected to directed or random chemical modifications, such as acylation, alkylation, esterification, and amidification, to produce structural analogs.

In certain embodiments, methods of the present invention comprise administering a therapeutically effective amount of a therapeutic agent as described above. The phrase “therapeutically effective amount,” as used herein, refers to an amount that kills or inhibits (partially or completely) growth of cervical cancer cells (e.g., HPV infected cells). The dose of a therapeutic agent administered to an individual in need of treatment will vary and will be determined for each individual with reference to, for example, the compound used, the route of administration, and the physical condition and body size of the individual. The daily dosage may be administered as a single dosage or may be divided into multiple doses. Actual dosage levels of a therapeutic agent may be varied so as to obtain amounts at the site of target cells (e.g., cervical cancer cells), effective to obtain the desired therapeutic or prophylactic response.

In certain embodiments, the subject methods of the invention can be used alone. Alternatively, the subject methods may be used in combination with other anti-viral or anti-cancer therapeutic approaches (e.g., administration of an anti-viral or anti-cancer agent, radiation therapy, phototherapy or immunotherapy) directed to treatment or prevention of cervical cancer or virus infections. For example, such methods can be used in prophylactic cancer prevention, prevention of cancer recurrence and metastases after surgery, and as an adjuvant of other traditional cancer therapy. Similarly, the subject methods of the invention may be combined with other antiviral therapies.

Thus, the subject methods of the invention may further include as optional ingredients one or more agents already known for their use in the inhibition of cervical cancer, for added clinical efficacy. These agents include, but are not limited to, interleukin-2, 5′-fluorouracil, nedaplatin, methotrexate, vinblastine, doxorubicin, carboplatin, paclitaxel (Taxol), cisplatin, 13-cis retinoic acid, pyrazoloacridine, vinorelbine, artemisinin, and artemisinin analogs. Appropriate amounts in each case will vary with the particular agent, and will be either readily known to those skilled in the art or readily determinable by routine experimentation. In other cases, the subject methods of the invention may further include as optional ingredients one or more agents already known for their anti-viral effects, for added clinical efficacy. These agents include, but are not limited to, 5′-fluorouracil, interferon alpha, imiquimod, landvudine, arsenic trioxide, capsaicin, nucleoside analogues (e.g., acyclovir), and antiviral vaccines.

The present invention also contemplates therapeutic agents obtainable from the screening methods described as below.

### Drug Screening Assays

There are numerous approaches to screening for therapeutic agents in cervical cancer therapy, which target one or more of the biomarkers (e.g., hTERT, IGFBP-3, transferrin receptor, beta-catenin or Myc-HPV E6 interaction). For example, high-throughput screening of compounds or molecules can be carried out to identify agents or drugs which inhibit cervical cancer. Test agents to be assessed can be any chemical (element, molecule, compound, drug), made synthetically, made by recombinant techniques or isolated from a natural source. For example, test agents can be peptides, polypeptides, peptoids, sugars, hormones, or nucleic acid molecules (such as antisense or RNAi nucleic acid molecules). In addition, test agents can be small molecules or molecules of greater complexity made by combinatorial chemistry, for example, and compiled into libraries. These libraries can comprise, for example, alcohols, alkyl halides, amines, amides, esters, aldehydes, ethers and other classes of organic compounds. Test agents can also be natural or genetically engineered products isolated from lysates or growth media of cells (e.g., bacterial, animal or plant), or can be the cell lysates or growth media themselves. Presentation of test compounds to the test system can be in either an isolated form or as mixtures of compounds, especially in initial screening steps.

In one embodiment, the present invention provides assays to screen for compounds that specifically inhibit protein-protein interaction (e.g., binding of Myc to HPV E6). To illustrate, such compounds can be identified by inhibition of binding of labeled Myc to HPV E6-Fc fusion protein. Compounds identified through this screening can then be tested in animal models of cervical cancer to assess their anti-tumor activity in vivo. An assay to identify a substance which interferes with interaction between Myc and HPV E6 can be performed with the component (e.g., cells, purified protein, including fusion proteins and portions having binding activity) which is not to be in competition with a test compound, linked to a solid support. The solid support can be any suitable solid phase or matrix, such as a bead, the wall of a plate or other suitable surface (e.g., a well of a microtiter plate), column pore glass (CPG) or a pin that can be submerged into a solution, such as in a well. Linkage of cells or purified protein to the solid support can be either direct or through one or more linker molecules.

In certain cases of the assays, an isolated or purified protein (e.g., a Myc polypeptide) can be immobilized on a suitable affinity matrix by standard techniques, such as chemical cross-linking, or via an antibody raised against the isolated or purified protein, and bound to a solid support. The matrix can be packed in a column or other suitable container and is contacted with one or more compounds (e.g., a mixture) to be tested under conditions suitable for binding of the compound to the protein. For example, a solution containing compounds can be made to flow through the matrix. The matrix can be washed with a suitable wash buffer to remove unbound compounds and non-specifically bound compounds. Compounds which remain bound can be released by a suitable elution buffer. For example, a change in the ionic strength or pH of the elution buffer can lead to a release of compounds. Alternatively, the elution buffer can comprise a release component or components designed to disrupt binding of compounds (e.g., one or more ligands or receptors, as appropriate, or analogs thereof which can disrupt binding or competitively inhibit binding of test compound to the protein).

In other embodiments, the present invention provides assays for screening for compounds that decrease or block the expression level (protein or nucleic acid) of a biomarker (hTERT, IGFBP-3, transferrin receptor or beta-catenin). Methods of detecting and optionally quantitating proteins can be achieved by techniques such as antibody-based detection assays. In these cases, antibodies may be used in a variety of detection techniques, including enzyme-linked immunosorbent assays (ELISAs), immunoprecipitations, and Western blots.

On the other hand, methods of detecting and optionally quantitating nucleic acids generally involve preparing purified nucleic acids and subjecting the nucleic acids to a direct detection assay or an amplification process followed by a detection assay. Amplification may be achieved, for example, by polymerase chain reaction (PCR), reverse transcriptase (RT), and coupled RT-PCR. Detection of nucleic acids is generally accomplished by probing the purified nucleic acids with a probe that hybridizes to the nucleic acids of interest, and in many instances, detection involves an amplification step as well. Northern blots, dot blots, microarrays, quantitative PCR, and quantitative RT-PCR are all well known methods for detecting nucleic acids.

Numerous assays for the detection and measurement of gene expression products are known and can be adapted for the determination of the level of expression of genes of interest disclosed in the present invention. For example, useful techniques for measuring the level of expression of a gene of interest in a cell sample include hybrid capture technique (see, for example, WO 93/10263, Digene); PCR in situ hybridization techniques (see, for example, Nuovo, 1997, Int J Cancer. 71:1056-60); branched DNA assays (see, for example, Chernoff et al., 1997, J Clin Microbiol. 35:2740-4); transcription-mediated amplification (TMA) (see, for example, Stoflet et al., 1988, Science. 239:491-4); and polymerase chain reaction (PCR), ligase chain reaction (LCR), self-sustained sequence replication (3SR), nucleic acid sequence based amplification (NASBA), strand displacement amplification (SDA), and amplification with Qβ replicase (see, for example, Birkenmeyer and Mushahwar, 1991, J Virol Methods. 35:117-26; Landegren, 1993, Bioessays. 15:761-5).

In some cases, one or more compounds can be tested simultaneously. Where a mixture of compounds is tested, the compounds selected by the foregoing processes can be separated (as appropriate) and identified by suitable methods (e.g., PCR, sequencing, chromatography). Large combinatorial libraries of compounds (e.g., organic compounds, peptides, nucleic acids) produced by combinatorial chemical synthesis or other methods can be tested (see e.g., Ohlmeyer, M. H. J. et al., Proc. Natl. Acad. Sci. USA 90:10922-10926 (1993) and DeWitt, S. H. et al., Proc. Natl. Acad. Sci. USA 90:6909-6913 (1993), relating to tagged compounds; see also, Rutter, W. J. et al., U.S. Pat. No. 5,010,175; Huebner, V. D. et al., U.S. Pat. No. 5,182,366; and Geysen, H. M., U.S. Pat. No. 4,833,092). Where compounds selected from a combinatorial library by the present method carry unique tags, identification of individual compounds by chromatographic methods is possible. Where compounds do not carry tags, chromatographic separation, followed by mass spectrophotometry to ascertain structure, can be used to identify individual compounds selected by the method, for example.

### Pharmaceutical Compositions

In certain embodiments, therapeutic agents (compounds) of the present invention are formulated with a pharmaceutically acceptable carrier. Therapeutic agents of the present invention can be administered alone or as a component of a pharmaceutical formulation (composition). The compounds may be formulated for administration in any convenient way for use in human medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compounds include those suitable for oral/ nasal, topical, parenteral and/or intravaginal administration. The formulations may conveniently be presented in unit dosage form and may be prepared by any methods well known in the art of pharmacy. The amount of active ingredient which can be combined with a carrier material to produce a single dosage form will vary depending upon the individual being treated and the particular mode of administration. The amount of active ingredient which can be combined with a carrier material to produce a single dosage form can be an amount of the compound which produces a therapeutic effect. Alternatively, multiple doses can be taken by an individual.

Methods of preparing these formulations or compositions include combining one or more compounds with one or more carriers and, optionally, one or more accessory ingredients. For example, the formulations are prepared by combining a compound with a liquid carrier, or a finely divided solid carrier, or both, and then, if necessary, shaping the product.

Formulations of the compounds suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound as an active ingredient. A compound may also be administered as a bolus, electuary or paste.

In solid dosage forms for oral administration (capsules, tablets, pills, dragees, powders, granules, and the like), a compound is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of a compound include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In particular, methods of the invention can be administered topically, either to skin or to mucosal membranes such as those on the cervix and vagina. This offers the greatest opportunity for direct delivery to tumor with the lowest chance of inducing side effects. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and atone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration of a compound include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a therapeutic compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Injectable depot forms are made by forming microencapsule matrices of the compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Formulations of the compounds for intravaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Optionally, such formulations suitable for vaginal administration also include pessaries, tampons, creams, gels, pastes, foams or spray formulations containing such carriers as are known in the art to be appropriate.

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of diagnosing or aiding in the diagnosis of cervical cancer in a female, comprising analyzing the status of at least two biomarkers selected from the group consisting of: hTERT, IGFBP-3, transferrin receptor, beta-catenin, Myc-HPV E6 interaction, HPV E7, and telomere length, in cervical cells of the female.

2. The method of claim 1, wherein, if one of the at least two biomarkers is hTERT, then the expression level of hTERT is analyzed and increased expression level of hTERT relative to an appropriate control indicates that the female has cervical cancer or is at increased risk of developing cervical cancer.

3. The method of claim 1, wherein, if one of the at least two biomarkers is IGFBP-3, then the expression level of IGFBP-3 is analyzed and increased expression level of IGFBP-3 relative to an appropriate control indicates that the female has cervical cancer or is at increased risk of developing cervical cancer.

4. The method of claim 1, wherein, if one of the at least two biomarkers is transferrin receptor, then the expression level of transferrin receptor is analyzed and increased expression level of transferrin receptor relative to an appropriate control indicates that the female has cervical cancer or is at increased risk of developing cervical cancer.

5. The method of claim 1, wherein, if one of the at least two biomarkers is beta-catenin, then the level of beta-catenin in the cytoplasm and/or nucleus is analyzed and increased level of beta-catenin in the cytoplasm and/or nucleus relative to an appropriate control indicates that the female has cervical cancer or is at increased risk of developing cervical cancer.

6. The method of claim 1, wherein, if one of the at least two biomarkers is Myc-HPV E6 interaction, then the association between Myc and HPV E6 is analyzed and the association between Myc and HPV E6 indicates that the female has cervical cancer or is at increased risk of developing cervical cancer.

7. The method of claim 1, wherein, if one of the at least two biomarkers is HPV E7, then HPV E7 expression is analyzed and the presence of HPV E7 expression indicates that the female has cervical cancer or is at increased risk of developing cervical cancer.

8. The method of claim 1, wherein, if one of the at least two biomarkers is telomere length, then the telomere length is analyzed and increased telomere length relative to an appropriate control indicates that the female has cervical cancer or is at increased risk of developing cervical cancer.

9-16. (canceled)

17. A method of detecting immortalization of cervical cells in a female, comprising analyzing the status of at least two biomarkers selected from the group consisting of: hTERT, IGFBP-3, transferrin receptor, beta-catenin, Myc-HPV E6 interaction, HPV E7, and telomere length, in cervical cells of the female.

18-59. (canceled)

60. A method of classifying the grade of a cervical lesion for diagnostic and prognostic purpose in a female, comprising: (a) determining the status of at least two biomarkers in a cervical cell of a female to provide an individual biomarker diagnostic for cervical lesions, wherein the at least two biomarkers are selected from the group consisting of: hTERT, IGFBP-3, transferrin receptor, beta-catenin, Myc-HPV E6 interaction, HPV E7, and telomere length; (b) comparing the status of the at least two biomarkers from (a) with a biomarker reference panel; and (c) classifying a cervical lesion for the female by said comparison of (b).

61. The method of claim 60, wherein, if one of the at least two biomarkers is hTERT, then the status of the biomarker is the expression level of hTERT.

62. The method of claim 60, wherein, if one of the at least two biomarkers is IGFBP-3, then the status of the biomarker is the expression level of IGFBP-3.

63. The method of claim 60, wherein, if one of the at least two biomarkers is transferrin receptor, then the status of the biomarker is the expression level of transferrin receptor.

64. The method of claim 60, wherein, if one of the at least two biomarkers is beta-catenin, the status of the biomarker is the level of beta-catenin in the cytoplasm and/or nucleus.

65. The method of claim 60, wherein, if one of the at least two biomarkers is Myc-HPV E6 interaction, then the status of the biomarker is the association between Myc and HPV E6.

66. The method of claim 60, wherein, if one of the at least two biomarkers is HPV E7, then the status of the biomarker is the expression of HPV E7.

67. The method of claim 60, wherein, if one of the at least two biomarkers is telomere length, then the status of the biomarker is the telomere length.

68. The method of claim 60, wherein the biomarker reference panel comprises a constituent panel developed using cervical cancer, high grade cervical lesion, low grade cervical lesion, and control group populations.

69-72. (canceled)
